(19) United States
(12) Patent Application Publication
(10) Pub. No.: US 2004/0060882 A1
(43) Pub. Date: Apr. 1, 2004

(54) Storage rack for bedpans

(76) Inventor: Barbara J. Messel, Joliet, IL (US)

Correspondence Address: LAW OFFICE OF BRIAN R. RAYVE, P.O. Box 11586, Salt Lake City, UT, 84147-0586, US

(21) Appl. No.: 10/664,125

(22) Filed: Sep. 16, 2003

Related U.S. Application Data
(60) Provisional application No. 60/411,400, filed on Sep. 16, 2002.

(51) Int. Cl. A47F 5/08

(52) U.S. Cl. 211/87.01; 211/88.01

(57) ABSTRACT

A storage rack which mounts to walls to hold bedpans and a method for cleaning, sanitizing, and storing hospital devices used to temporarily contain bodily fluids and other bodily products. The storage rack includes a support for receiving and holding one or more bedpans, a urinal, graduated bottles, and a cleaning device in the form of a manual scrubbing device or a spray bottle. The storage rack removably mounts to a shield plate made of plexiglass, stainless steel, or similar sheet material which is mounted to the wall to protect it from splatters. The support and the shield are of a rectangular configuration fabricated from cut and bent metal rods. The support has respective upper and lower racks each of which includes a front wall, a pair of side walls, and a rear wall which define respective interior device receiving spaces. The upper and lower racks are interconnected by a plurality of connecting members also comprising metal rods. The upper rack is sized to receive the urinal and graduated bottles and the lower rack to receive the bedpans. The connecting members may be cut to separate the upper and lower racks, and the shield plate may be cut to provide respective upper and lower shield plates for the upper and lower racks which are mounted to the wall in a desired arrangement. The support includes respective upper and lower brackets externally affixed thereto and adapted to receive and hold the cleaning device.

### RELATED APPLICATION

This application claims priority of my copending U.S. Provisional Patent Application No. 60/411,400 filed Sep. 16, 2002, the entire disclosure of which is herein incorporated by reference thereto.

### BACKGROUND OF THE INVENTION

1. Field

The present invention generally relates to storage racks and cleaning methods for hospital devices, and more specifically to storage racks and cleaning methods for bedpans, urinals, graduated bottles, and other receptacles used to temporarily contain bodily fluids and other bodily products, and methods for cleaning, sanitizing, and storing such hospital receptacles.

2. State of the Art

Immobile patients in hospitals, other such health care facilities, and in-home care cannot walk or be easily taken by wheelchair to the bathroom. Consequently, various receptacles have been developed to receive urine and feces such as bedpans and urinals while the patient remains in bed. Likewise, various urine measuring receptacles are utilized such as disposable foley bags which are emptied into graduated bottles to measure urine output. While these receptacles are generally adequate for their intended purposes, cleaning and storage thereof has remained a problem. There is typically no designated area for storage of such receptacles when not in use. The receptacles must be located close to the patients to allow immediate access due to the oftentimes sudden need to use such receptacles based on patient needs. Yet the receptacles cannot significantly interfere with care for the patients. Sometimes such receptacles are temporarily or permanently stored on over-bed stands or on bedside cabinets. However, oftentimes the receptacles are missing when needed or are accidentally knocked over spilling the bodily products when stored in such locations. Likewise, there is typically no standard way of cleaning and sanitizing the receptacles following use and emptying of the bodily materials. The receptacles are washed by hand using whatever cleaning device is convenient using standard hospital cleaning and disinfecting solutions. Oftentimes, no cleaning device is available conveniently available, requiring searching for such cleaning device or postponement of cleaning which may lead to a biohazard problem.

Several devices have been patented which attempt to solve certain aspects of the storage problem for receptacles. For example, in U.S. Pat. No. 4,573,653 issued to Boettger on Mar. 4, 1986 is disclosed a urinal holder for hospital over-bed tables. The urinal holder is primarily formed from a single, continuous piece of wire which is formed to provide an upper urinal receiving opening and a lower base upon which a urinal may rest. Hooks disposed at an upper rear portion of the urinal holder are used to attach the holder to an upright post of the over-bed table. The rear of the base also engages the upright post to further stabilize the urinal holder. In U.S. Pat. No. 5,626,318 issued to Boettger, et al. on May 6, 1997 is disclosed a receptacle holder assembly for urinals which is mountable on a variety of differently configured support surfaces. The assembly includes a holder unit having a pair of spaced apart mounting legs for supporting a urinal. An attachment member is coupled to each leg for independent pivoting movement thereof about the respective leg. Each holder is provided with either mating hook and loop strips or adhesives allowing the receptacle holder assembly to be secured to the support surfaces. Both of these devices accommodate only a single urinal.

Several other devices have been marketed which also attempt to solve certain aspects of the storage problem for receptacles. A recessed bedpan and urinal cabinet is sold by American Specialties, Inc. of Yonkers, N.Y. which accommodates a bedpan and one or more urinals. The cabinet has a single shelf which supports the bedpan and a closeable door. The cabinet requires cutting out a piece of wall in which to mount the cabinet, and must be mounted between vertical wall studs or the studs be cut. The same company sells a surface mounted bedpan and urinal rack. The rack includes a wall mounted plate and a pair of brackets or shelves which respectively accommodate a single bedpan and urinal. The rack does not accommodate storage of multiple urinals.

There is a need for a storage device and cleaning method for hospital receptacles which allows storage of bedpans, multiple urinals, and other receptacles. The storage device would not require cutting of walls, be locatable closely adjacent patients, and accommodate a cleaning device.

### SUMMARY OF THE INVENTION

The present invention is a storage rack for bedpans which mounts to a vertical surface, and a method for cleaning, sanitizing, and storing hospital devices used to temporarily contain bodily fluids and other bodily products.

The storage rack for bedpans comprises a support which is adapted to receive and hold at least one bedpan, and a shield which is adapted to be mounted to the vertical surface to protect it from splatters. The support is adapted to be mountable to the shield to retain the bedpan on the vertical surface.

In a first preferred storage rack for bedpans, the support and the shield are of substantially rectangular configuration fabricated substantially from rods. The support comprises an upper rack and a lower rack which are interconnected by at least one connecting member comprising a rod. The lower rack is adapted to receive and hold the bedpan. The upper and lower racks each include a front wall, a pair of side walls, and a rear wall which define respective interior device receiving spaces. The interior device receiving space of the lower rack comprises a bedpan receiving space in which the urinal may be placed. Each connecting member is adapted to be cut to separate the upper and lower racks. The shield plate is also adapted to be cut to provide respective upper and lower shield plates for the upper and lower racks. The upper and lower shield plates are mounted to the vertical surface in a desired arrangement and with the upper and lower racks respectively attached thereto. Alternatively, the upper and lower racks and the upper and lower shields or shield plates are initially fabricated as separate components which assemble together in the same manner.

In a second preferred storage rack for bedpans, the support is removably mountable to the shield plate. The support includes a plurality of the mounting plates affixed thereto, each having the keyhole therethrough with a circular bottom and a narrower slot extending therefrom. The shield includes a plurality of the headed studs arranged in corresponding positions to the keyholes. Each headed stud has a rod affixed extending from the shield and a head spaced from the shield. The rods are of such a size as to closely slidably fit through the slots and the heads are of such a size as to closely pass through the circular bottoms of the keyholes. The shield comprises a shield plate made of a sheet material chosen from the group consisting of plexiglass, stainless steel, aluminum, and plastic, the shield plate. The shield has a plurality of mounting holes therethrough for passing respective screws to mount the shield plate to the vertical surface.

In a third preferred storage rack for bedpans, the support is adapted to receive and hold at least one device in addition to the bedpan chosen from the group consisting of a urinal and a graduated bottle. The storage rack further comprises a cleaning device chosen from the group consisting of a manual urinal scrubbing device and a spray bottle, and further comprises a container adapted to receive and hold the cleaning device. The support includes respective upper and lower brackets externally affixed thereto and adapted to receive and hold the cleaning device.

The method for cleaning, sanitizing, and storing hospital devices used to temporarily contain bodily fluids and other bodily products, comprises the steps of: 1) providing a support mounted on a vertical surface adapted for receiving and supporting a cleaning device and at least one hospital device; 2) placing the cleaning device and the hospital device on the support; 3) removing at least one hospital device from the support and using it to receive the bodily product; 4) dumping the bodily product from the hospital device; 5) removing the cleaning device from the support device; 6) cleaning and sanitizing the hospital device using the cleaning device; and 7) replacing the cleaning device and the cleaned hospital device on the support for later use.

In a first preferred method, the support provided is mounted on the vertical surface with a shield therebetween to protect the vertical surface from splatters. The hospital device is a bedpan and at least one other chosen from the group consisting of a urinal and a graduated bottle.

A second preferred method further comprises the step of providing a cleaning and disinfecting solution for use with the cleaning device. The cleaning device used is chosen from the group consisting of a manual scrubbing device and a spray bottle, used with a container adapted to receive and hold the cleaning device on the support.

### THE DRAWINGS

The best mode presently contemplated for carrying out the invention is illustrated in the accompanying drawings, in which:

FIG. 1 is a front elevational view of a storage rack of the present invention which includes a shield plate permanently mounted to a wall and a wire rack removably mounted thereto holding a bedpan, a urinal, a plurality of graduated bottles, a bedpan cleaning device comprising a hand mop, and a container for holding cleaning solution;

FIG. 2, a side elevational view of the storage rack mounted to the wall taken on the line 2-2 of FIG. 1 showing the hand mop and container retained by a pair of brackets;

FIG. 3, a front elevational view of the wire rack;

FIG. 4, a side elevational view of the wire rack taken on the line 4-4 of FIG. 3;

FIG. 5, a view side elevational view of the wire rack taken on the line 5-5 of FIG. 3 showing the pair of brackets for retaining the hand mop and container;

FIG. 6, a top plan view of the brackets taken on the line 6-6 of FIG. 3;

FIG. 7, a top plan view of the wire rack taken on the line 7-7 of FIG. 3;

FIG. 8, a front elevational view of the shield plate;

FIG. 9, a front elevational view of a mounting plate affixed to the wire rack taken on the line 9-9 of FIG. 3 to a much enlarged scale showing the keyhole therethrough;

FIG. 10, a side elevational view of the mounting plate taken on the line 10-10 of FIG. 1 to a much enlarged scale;

FIG. 11, a front elevational view of a modified storage rack with the wire rack as cut at the dotted lines of FIG. 3 and the shield plate as cut at the dotted lines of FIG. 8, respective pieces of the of the shield plate being permanently mounted to a wall side-by-side and respective pieces of the cut wire rack being removably mounted thereto holding the bedpan, the urinal, the plurality of graduated bottles, and a bedpan cleaning device comprising a spray bottle containing cleaning solution, and a container for holding the spray bottle; and

FIG. 12, a perspective view of a protective cap used to cover respective ends of wires cut to make the cut wire rack.

### DETAILED DESCRIPTION OF THE ILLUSTRATED EMBODIMENTS

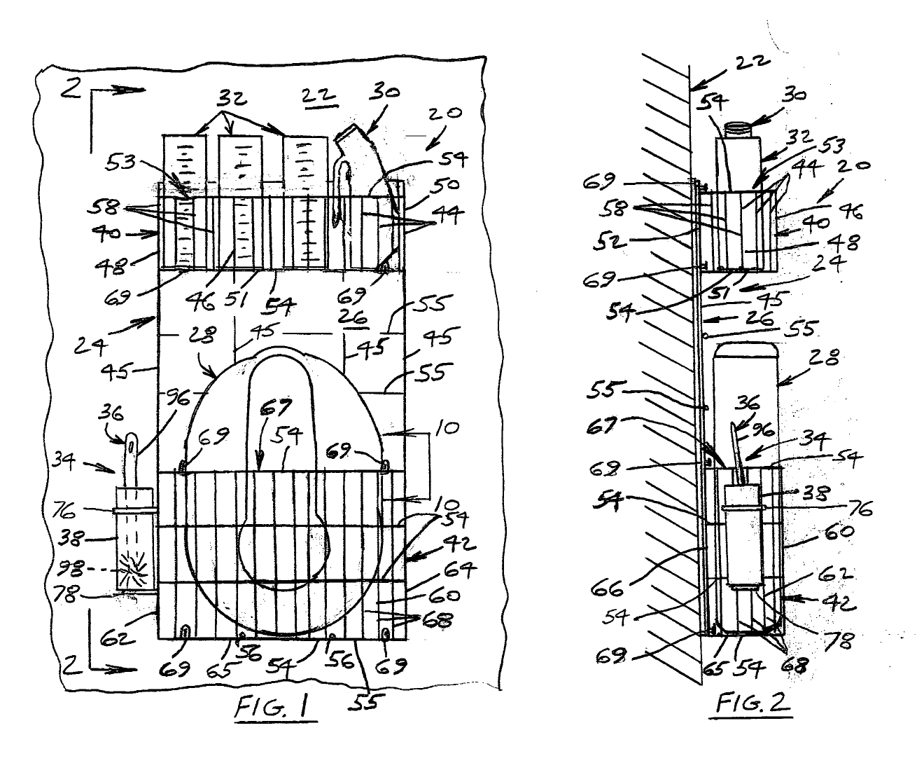

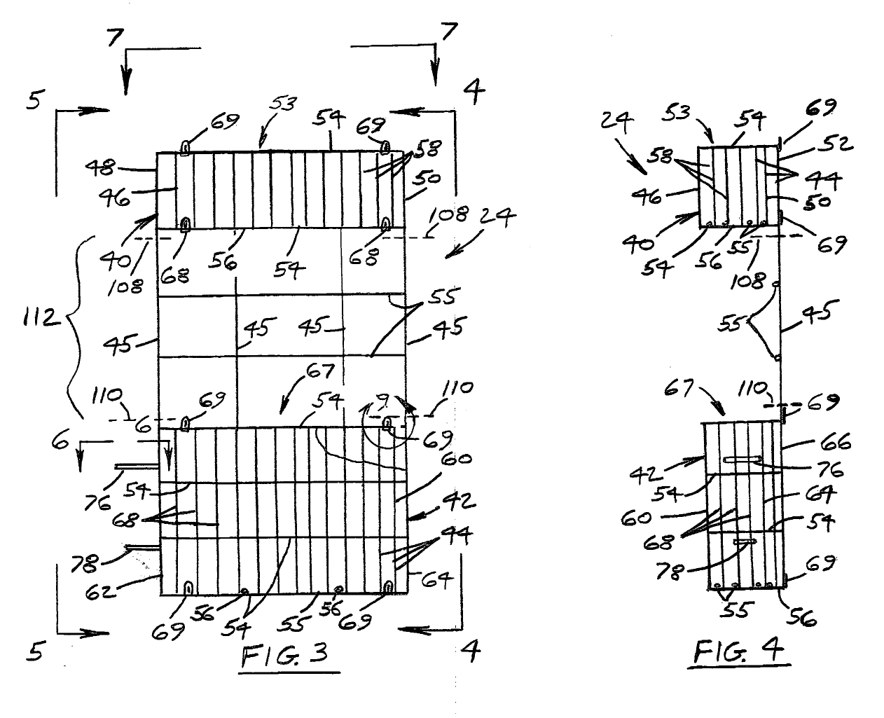

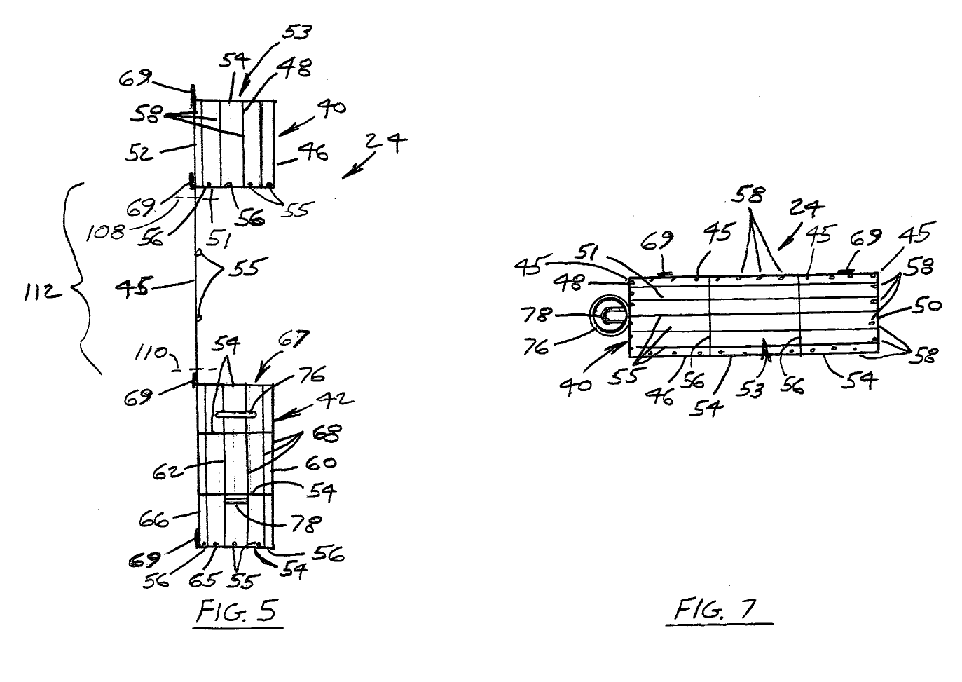

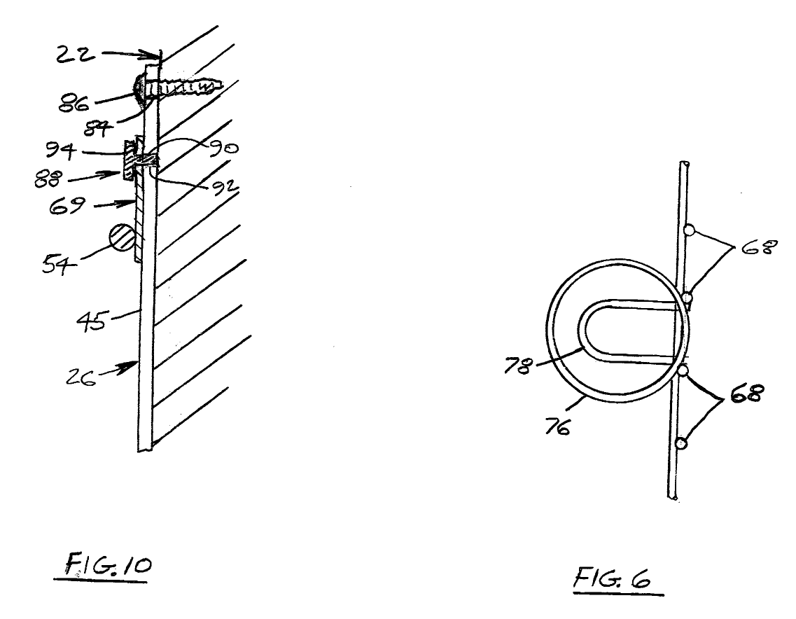

Referring to FIGS. 1-10, therein is shown a storage rack for bedpans and other hospital receptacles used to temporarily contain bodily fluids and other bodily products in accordance with the present invention, designated generally at 20. The storage rack 20 mounts to a vertical surface such as that of a wall 22 comprising an open stud wall covered with wallboard, a cinder block wall, a concrete wall, or other such wall construction.

The storage rack 20 includes a support which is fabricated substantially from rods, in the form of a wire rack 24 which is adapted to be removably mountable to a shield in the form of a shield plate 26 permanently or semi-permanently mounted to the wall 22. The wire rack 24 and shield plate 26 are each substantially of rectangular configuration. The storage rack 20 is adapted to receive and hold at least one hospital receptacle such as a bedpan 28, a urinal 30, a plurality of graduated bottles 32. Additionally, the storage rack 20 is adapted to receive and hold a first bedpan cleaning device 34 in the form of a manual scrubbing device or hand mop 36, and a cylindrical container 38 for holding cleaning solution (not shown) to retain the receptacles and the hand mop 36 on the wall 22. The shield plate 26 is adapted to protect the wall 22 from splatters of urine, feces, and other bodily products which may be contained in the receptacles such as if cleaning is not done prior to storage thereof in storage rack 20.

The wire rack 24 is in the form of a unitized body which includes an upper rack 40 and a lower rack 42 which are fabricated from a plurality of cut pieces of raw thick wire or raw rod 44 which are bent to shape and affixed together in a spaced, parallel relationship such as by welding, adhesives, or other such fastening technology at mutual contact points to form an open framework which facilitates airflow and drying of the bedpan 28, the urinal 30, and the plurality of graduated bottles 32. The raw rod 44 is a relatively slender, continuous length of a suitable plastic material, or a metal material such as stainless steel, carbon steel, or aluminum having a uniform diameter throughout its length. If metal rods are used, the entire assembled wire rack 24 may thereafter be dip or spray coated with an outer layer (not shown) of a suitable plastic material such as vinyl which is durable and corrosion-resistant and which may be colored as desired to enhance the aesthetic appeal of the wire rack. Alternatively, the entire assembled wire rack 24 may be plated with a suitable metal coating such as chrome or nickel, or painted to minimize rust and corrosion.

A plurality of common connecting members or rods 45 interconnect the upper rack 40 and the lower rack 42. The connecting rods 45 are adapted to be cut to separate the upper rack 40 from the lower rack 42 by being of a material and size which is cuttable using wire cutters or the like, having breakable serrations (not shown), or similar arrangement. The upper rack 40 includes a front wall 46, a pair of side walls 48 and 50, a bottom wall 51, and a rear wall 52 which includes portions of the connecting rods 45. The walls 46, 48, 50, 51, and 52 define a device receiving space 53 in which the urinals 30 and graduated bottles 32 may be placed. The walls 46, 48, 50, 51, and 52 include bent rods 54, transverse rods 55, longitudunal rods 56, and short vertical rods 58. The lower rack 42 includes a front wall 60, a pair of side walls 62 and 64, a bottom wall 65, and a rear wall 66 which also includes portions of the connecting rods 45. The walls 60,62, 64, and 66 define a bedpan receiving space 67 in which the bedpan 28 may be placed. The walls 60, 62, 64, 65, and 66 include the bent rods 54, the transverse rods 55, the longitudinal rods 56, and long vertical rods 68. A plurality of mounting plates 69 are affixed to upper rack 40 and lower rack 42, each having a keyhole 70 therethrough with a circular bottom 72 and a narrower slot 74 extending therefrom. Lower rack 42 includes respective upper and lower brackets 76 and 78 which are externally affixed to the side wall 62 to receive and hold the hand mop 36 and container 38. The upper bracket 76 comprises a ring of the raw wire rod 44 adapted to closely receive the container 38, and the lower bracket comprises a platform or oval of the raw wire rod 44 adapted to vertically support the container 38.

The shield plate 26 is of a rectangular shape slightly taller than the wire rack 24 made of a suitable cleanable sheet material such as plexiglass, stainless steel, or aluminum to protect the wall 22 from splatters. Suitable sheet materials include plexiglass of a thickness of between about 0.125 and 0.187 inch thick, stainless steel of a thickness of between about 0.062 and 0.125 inch thick, and aluminum of a thickness of between about 0.125 and 0.250 inch thick. The shield plate 26 is adapted to be cut by being of a material and thickness such as stated which may be readily cut using an electric hand saw or the like. The shield plate 26 has a plurality of mounting holes 84 therethrough for passing respective screws 86 for mounting to the wall 22. A plurality of headed studs 88 are arranged in corresponding positions to the keyholes 70, each having a rod 90 affixed extending from respective stud holes 92 of the shield plate 26 such as at threaded, welded, or pressfit connections. Each headed stud terminates at a head 94 disposed slightly spaced from the shield plate 26. The rods 90 are of such a size as to closely slidably fit through the slots 74 and the heads 94 being of such a size as to closely pass through the circular bottoms 72 of keyholes 70. The stud holes 92 with protruding headed studs 88 correspond in position to the keyholes 70 through the mounting plates 69 of wire rack 24 such that the wire rack 24 is removably affixable to the shield plate 29 supported on the headed studs 88. This is accomplished by first aligning corresponding pairs of headed studs 88 and keyholes 70, then inserting the heads 94 through the circular bottoms 72 of keyholes 70, and finally lowering the wire rack 24 such that the rods 90 slide along the slots 74 thereof. The procedure is reversed to remove wire rack 24 from shield plate 26.

The hand mop 36 and the container 38 adapted to receive and hold the hand mop 36 with cleaning solution are of conventional design used for cleaning and disinfecting such as in hospitals. The hand mop 36 includes an elongate handle 96 of plastic or similar cleanable material to which an absorbent ball 98 of string or similar absorbent material is attached. The container 38 is made of plastic or similar cleanable material.

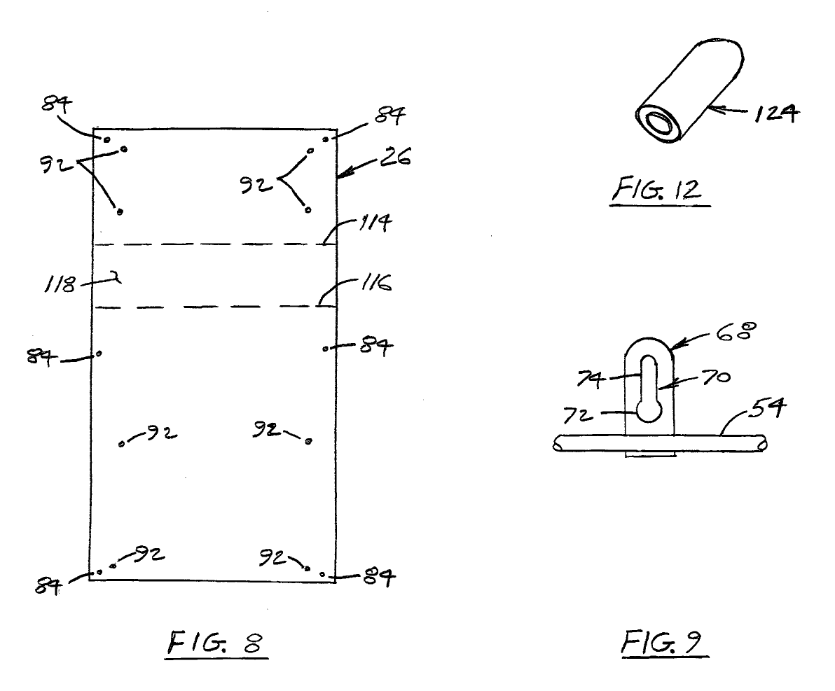

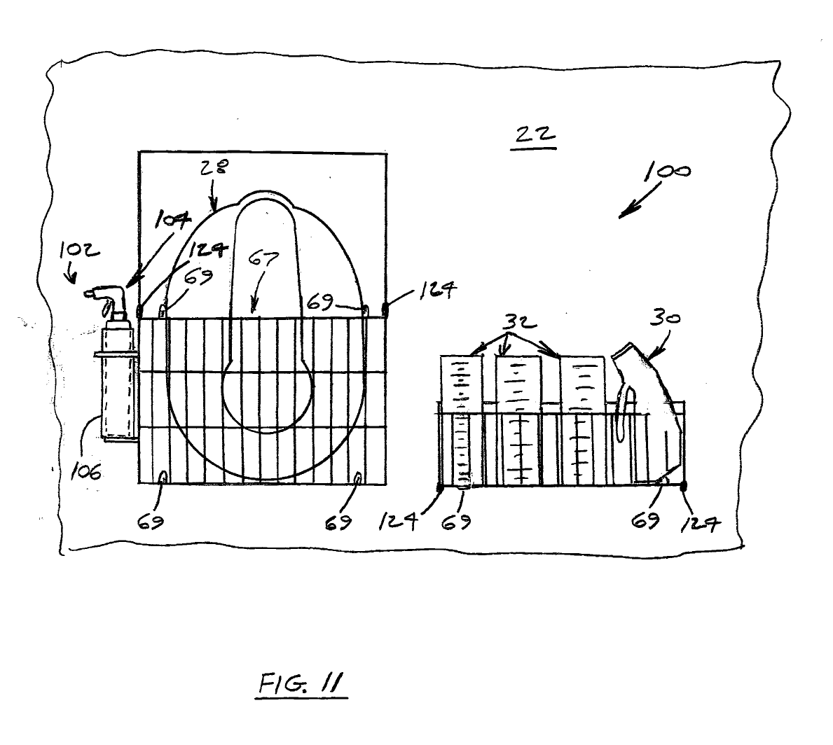

Referring to FIGS. 11 and 12, therein is shown a modified storage rack 100 for bedpans 28 which mounts to the wall 22. The storage rack 100 is made by cutting the storage rack 20 to separate the upper rack 40 from the lower rack 42, and cutting the shield plate 26 which is permanently mounted to the wall 22. The storage rack 100 is shown supporting the bedpan 28, the urinal 30, the plurality of graduated bottles 32, and a second bedpan cleaning device 102 in the form of a spray bottle 104 for holding cleaning solution (not shown), and a cylindrical container 106 for holding the spray bottle 104.

The storage rack 20 is cut to produce the storage rack 100 by cutting through the connecting rods 45 which interconnect the upper rack 40 and the lower rack 42 on dotted cutting lines 108 and 110 to remove respective middles 112 of connecting rods 45. The shield plate 26 is cut on dotted cutting lines 114 and 116 to remove a middle 118 thereof forming respective upper and lower shield plates 120 and 122 for the upper and lower racks 40 and 42. The upper and lower shield plates 120 and 122 are mounted to the wall 22, using the screws 86, in a side-by-side or other desired arrangement and with the upper and lower racks 40 and 42 respectively attached thereto. The separated upper and lower racks 40 and 42 are attached to respective of the upper and lower shield plates 120 and 122 as stated above. A plurality of resilient protective caps 124 are used to cover respective remaining ends 126 and 128 of the cut connecting rods 45 to prevent injury to persons brushing there against. Alternatively, the upper and lower racks 40 and 42, and the upper and lower shield plates 120 and 122 can be initially made as separate components.

The second bedpan cleaning device 102 including the spray bottle 104 for holding cleaning solution and container 106 for holding the spray bottle 104 are of conventional design used for cleaning and disinfecting such as in hospitals. The spray bottle 104 includes a trigger operable pump spray head 130 of plastic or similar cleanable material to which a bottle 132 of plastic or similar material is screw attached.

A method for cleaning, sanitizing, and storing hospital devices used to temporarily contain bodily fluids and other bodily products, comprising the steps of: 1) providing a support mounted on a vertical surface adapted for receiving and supporting a cleaning device and at least one hospital device; 2) placing the cleaning device and the hospital device on the support; 3) removing at least one hospital device from the support and using it to receive the bodily product; 4) dumping the bodily product from the hospital device; 5) removing the cleaning device from the support device; 6) cleaning and sanitizing the hospital device using the cleaning device; and 7) replacing the cleaning device and the cleaned hospital device on the support for later use.

The support provided may be mounted on the vertical surface with a shield therebetween to protect the wall from splatters, and the hospital device may comprise a bedpan and at least one other chosen from the group consisting of a urinal and a graduated bottle. The method may further comprise the step of providing a cleaning and disinfecting solution for use with the cleaning device, and the cleaning device used chosen from the group consisting of a manual scrubbing device and a spray bottle, used with a container adapted to receive and hold the cleaning device on the support.

Many variations to the present invention are possible while staying within the same inventive concept. For example, the support may be fixedly mounted to the shield plate. The raw rods may be of other than cylindrical cross-section such as square or hex-shaped. The raw rod construction may be replaced by plastic panels molded such as by pressure molding or injection molding. Other connecting devices other than headed studs and mating keyholes may be used.

Whereas this invention is here illustrated and described with reference to embodiments thereof presently contemplated as the best mode of carrying out such invention in actual practice, it is to be understood that various changes may be made in adapting the invention to different embodiments without departing from the broader inventive concepts disclosed herein and comprehended by the claims that follow.

I claim:

1. A storage rack for bedpans which mounts to a vertical surface, comprising:
a support adapted to receive and hold at least one bedpan;
a shield adapted to be mounted to the vertical surface to protect the vertical surface from splatters; and
wherein said support is adapted to be mountable to said shield to retain the bedpan on the vertical surface.

2. The storage rack according to claim 1, wherein the support is removably mountable to the shield plate.

3. The storage rack according to claim 2, wherein the support includes a plurality of the mounting plates affixed thereto, each having the keyhole therethrough with a circular bottom and a narrower slot extending therefrom, and the shield includes a plurality of the headed studs arranged in corresponding positions to said keyholes, each having a rod affixed extending from said shield and a head spaced from said shield, said rods being of such a size as to closely slidably fit through said slots and said heads being of such a size as to closely pass through said circular bottoms of keyholes.

4. The storage rack according to claim 1, wherein the shield comprises a shield plate made of a sheet material chosen from the group consisting of plexiglass, stainless steel, and aluminum, said shield plate having a plurality of mounting holes therethrough for passing respective screws to mount said shield plate to the vertical surface.

5. The storage rack according to claim 1, wherein the support includes a front wall, a pair of side walls, and a rear wall which define a bedpan receiving space in which the bedpan may be placed.

6. The storage rack according to claim 1, wherein the support comprises respective upper and lower racks which are interconnected by at least one connecting member.

7. The storage rack according to claim 6, wherein the lower rack is adapted to receive and hold the bedpan.

8. The storage rack according to claim 6, wherein each connecting member is adapted to be cut to separate the upper and lower racks, and the shield plate is adapted to be cut to provide respective upper and lower shield plates for said upper and lower racks which are mounted to the vertical surface in a desired arrangement and with said upper and lower racks respectively attached thereto.

9. The storage rack according to claim 1, wherein the support is fabricated substantially from rods.

10. The storage rack according to claim 1, wherein the support is adapted to receive and hold at least one device additional to the bedpan chosen from the group consisting of a urinal, a graduated bottle, and a cleaning device.

11. The storage rack according to claim 10, further comprising:
a cleaning device chosen from the group consisting of a manual scrubbing device and a spray bottle;
a container adapted to receive and hold said cleaning device; and
wherein the support includes respective upper and lower brackets externally affixed thereto and adapted to receive and hold said cleaning device.

12. The storage rack according to claim 11, wherein the upper bracket comprises a ring adapted to closely receive cylindrical containers, and the lower bracket comprises a platform adapted to vertically support said container, both being substantially horizontally disposed.

13. The storage rack according to claim 1, wherein the support comprises separate upper and lower racks, and the shield comprises separate upper and lower shields for said upper and lower racks which are mounted to the vertical surface in a desired arrangement and with said upper and lower racks respectively attached thereto.

14. The storage rack according to claim 1, wherein the support and shield are each substantially of rectangular configuration.

15. A storage rack for bedpans which mounts to a vertical surface, comprising:
a support of substantially rectangular configuration fabricated substantially from rods adapted to receive and hold at least one bedpan, said support comprising an upper rack and a lower rack which are interconnected by at least one connecting member comprising a rod, said lower rack being adapted to receive and hold the bedpan, said upper and lower racks each including a front wall, a pair of side walls, and a rear wall which define respective interior device receiving spaces, said interior device receiving space of said lower rack comprising a bedpan receiving space in which the bedpan may be placed;
a shield of substantially rectangular configuration adapted to be mounted to the vertical surface to protect the vertical surface from splatters; and
wherein said support is adapted to be mountable to said shield to retain the bedpan on the vertical surface, each connecting member is adapted to be cut to separate the upper and lower racks, and said shield plate is adapted to be cut to provide respective upper and lower shield plates for said upper and lower racks which are mounted to the vertical surface in a desired arrangement and with said upper and lower racks respectively attached thereto.

16. The storage rack according to claim 15, wherein the support is removably mountable to the shield plate, said support which includes a plurality of the mounting plates affixed thereto, each having the keyhole therethrough with a circular bottom and a narrower slot extending therefrom, and the shield includes a plurality of the headed studs arranged in corresponding positions to said keyholes, each having a rod affixed extending from said shield and a head spaced from said shield, said rods being of such a size as to closely slidably fit through said slots and said heads being of such a size as to closely pass through said circular bottoms of keyholes, and said shield comprises a shield plate made of a sheet material chosen from the group consisting of plexiglass, stainless steel, aluminum, and plastic, said shield plate having a plurality of mounting holes therethrough for passing respective screws to mount said shield plate to the vertical surface.

17. The storage rack according to claim 15, wherein the support is adapted to receive and hold at least one device in addition to the bedpan chosen from the group consisting of a urinal and a graduated bottle, further comprising a cleaning device chosen from the group consisting of a manual scrubbing device and a spray bottle, further comprising a container adapted to receive and hold said cleaning device, and wherein said support includes respective upper and lower brackets externally affixed thereto and adapted to receive and hold said cleaning device.

18. A method for cleaning, sanitizing, and storing hospital devices used to temporarily contain bodily fluids and other bodily products, comprising the steps of:
providing a support mounted on a vertical surface adapted for receiving and supporting a cleaning device and at least one hospital device;
placing the cleaning device and the hospital device on the support;
removing at least one hospital device from the support and using it to receive the bodily product;
dumping the bodily product from the hospital device;
removing the cleaning device from the support device;
cleaning and sanitizing the hospital device using the cleaning device; and
replacing the cleaning device and the cleaned hospital device on the support for later use.

19. The method according to claim 18, wherein the support provided is mounted on the vertical surface with a shield therebetween to protect the vertical surface from splatters, and the hospital device is a bedpan and at least one other chosen from the group consisting of a urinal and a graduated bottle.

20. The method according to claim 18, further comprising the step of:
providing a cleaning and disinfecting solution for use with the cleaning device; and
wherein the cleaning device used is chosen from the group consisting of a manual scrubbing device and a spray bottle, used with a container adapted to receive and hold the cleaning device on the support.
